(19) United States
(12) Plant Patent Application Publication
(10) Pub. No.: US 2019/0069457 P1
(43) Pub. Date: Feb. 28, 2019

(54) Strawberry plant named 'Caprice'

(50) ;

(71) Applicant: Sweet Darling Sales, Inc., Aptos, CA (US)

(72) Inventor: John Larse, Watsonville, CA (US)

(21) Appl. No.: 15/998,932

(22) Filed: Aug. 23, 2018

Related U.S. Application Data
(60) Provisional application No. 62/550,183, filed on Aug. 25, 2017.

(51) Int. Cl. A01H 6/74

(52) U.S. Cl. PLT/208

(57) ABSTRACT

The present invention provides new and distinct strawberry plants designated as ‘Caprice’ (a.k.a. ‘109862’) and ‘Victor’ (a.k.a. ‘102850’).

### CROSS-REFERENCE TO RELATED APPLICATION

The present application claims priority to, and the benefit of U.S. Provisional Patent Application No. 62/550,183, filed on Aug. 25, 2017, which is herein incorporated by reference in its entirety for all purposes.

Latin name of the genus and species: Fragaria x ananassa.

Varietal denominations: ‘Caprice’ (a.k.a. ‘109862’). ‘Victor’ (a.k.a. ‘102850’).

### BACKGROUND OF THE INVENTION

The present invention relates to new and distinct strawberry plants designated as ‘Caprice’ (a.k.a. ‘109862’) and ‘Victor’ (a.k.a. ‘102850’).

‘Caprice’ (a.k.a. 109862) is the result of a controlled-cross between a female parent cultivar designated ‘109013’ and a male parent cultivar designated ‘Crystalina’ (U.S. Plant Pat. No. 25,867) made by the Inventor and was first fruited in Watsonville, Calif. growing fields. Following selection and during testing, the plant was originally designated ‘109862’ and subsequently named ‘Caprice’. ‘Caprice’ is a day-neutral plant.

‘Victor’ (a.k.a. ‘102850’) is the result of a controlled-cross between a female parent cultivar designated ‘1046’ and a male parent cultivar designated ‘1033’ made by the Inventor and was first fruited in Watsonville, Calif. growing fields. Following selection and during testing, the plant was originally designated ‘102850’ and subsequently named ‘Victor’. ‘Victor’ is a short-day plant.

These new strawberry plants were asexually reproduced via runners (stolons) by the inventor at Watsonville, Calif. Asexual propagules from the original source have been tested in Watsonville growing fields and to a limited extent, grower fields in high elevation. The properties of these plants were found to be transmissible by such asexual reproduction. These plants are stable and reproduces true to type in successive generations of asexual reproduction.

### BRIEF SUMMARY OF THE INVENTION

This invention relates to new and distinctive strawberry plants designated as ‘Caprice’ (a.k.a. ‘109862’) and ‘Victor’ (a.k.a. ‘102850’). These plants are primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry plant ‘Caprice’ and ‘Victor’ from known strawberry plants. In addition, these plants were confirmed, or will be confirmed to be a unique strawberry germplasm using Short Sequence Repeats (SSRs). Plants for the botanical measurements in the present application were grown as annuals. Any color references are made to The Royal Horticultural Society Colour Chart, 1995 Edition, except where general terms of ordinary dictionary significance are used.

When compared to the proprietary male parent, ‘Caprice’ has smaller fruit than its male parent. Additionally, ‘Caprice’ produces more fruit and is a larger plant than the male parent. When compared to the proprietary female parent, ‘Caprice’ is a larger, more upright plant than the female parent. Additionally, ‘Caprice’ has longer petioles and larger fruit than the female parent.

When ‘Victor’ is compared to the proprietary male parent, ‘Victor’ has larger fruit. When compared to the proprietary female parent, ‘Victor’ presents larger, more conical fruit. Additionally, the female parent presents more firm fruit than ‘Victor’.

### DESCRIPTION OF THE DRAWINGS

The accompanying color photographs depict various characteristics of the cultivars as nearly true as possible to make color reproductions.

FIG. 1A shows ‘Caprice’ plant about 6-month old.

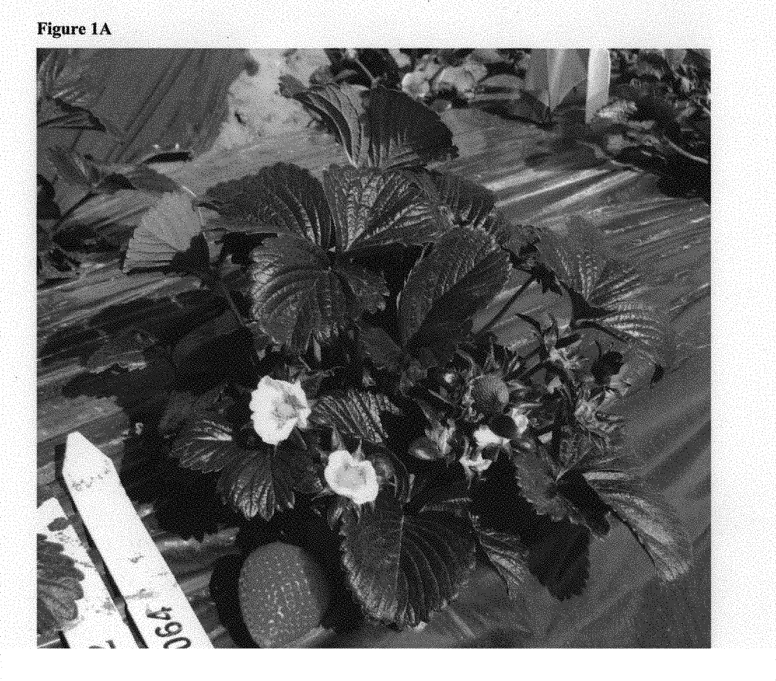

FIG. 1B shows ripe and near-ripe fruits of ‘Caprice’ about 8-month old.

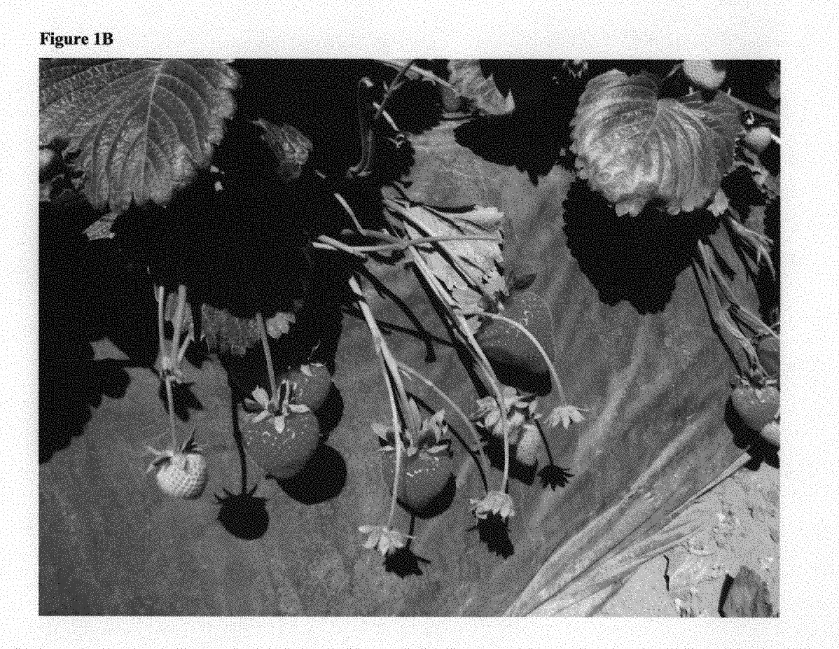

FIG. 1C shows flowers of ‘Caprice’.

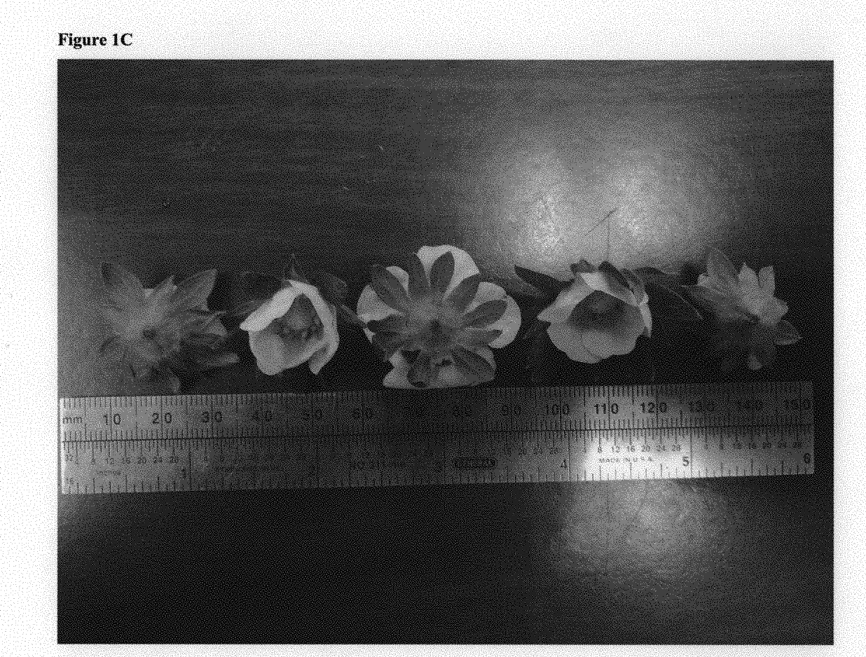

FIG. 1D shows fruits of ‘Caprice’.

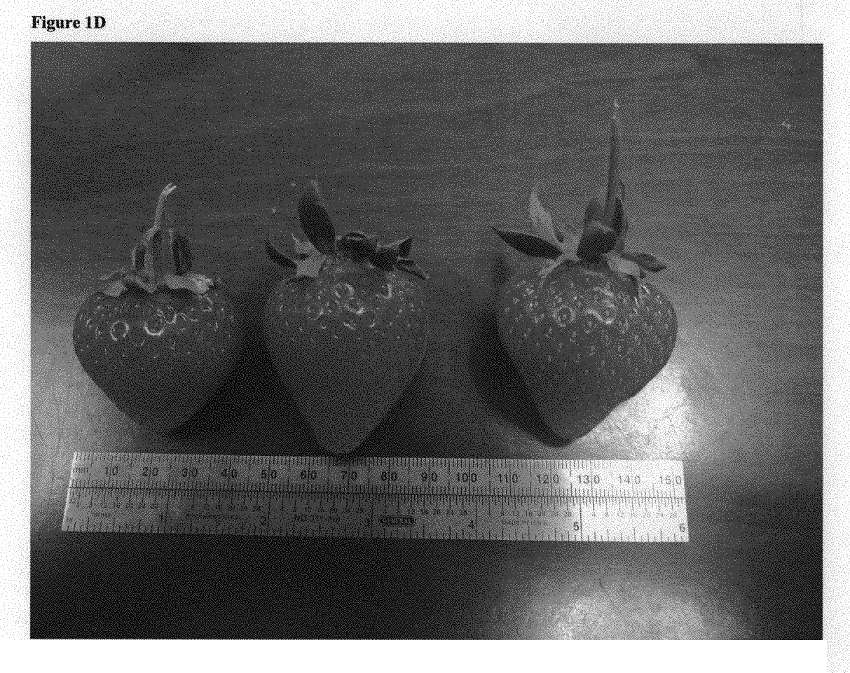

FIG. 1E shows cut fruits of ‘Caprice’.

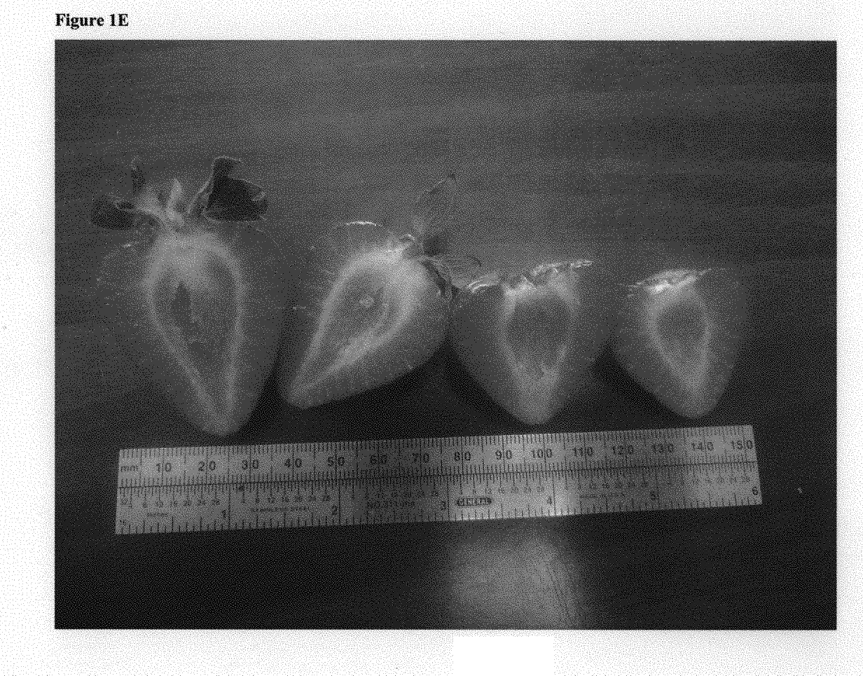

FIG. 1F shows upper leaf of ‘Caprice’.

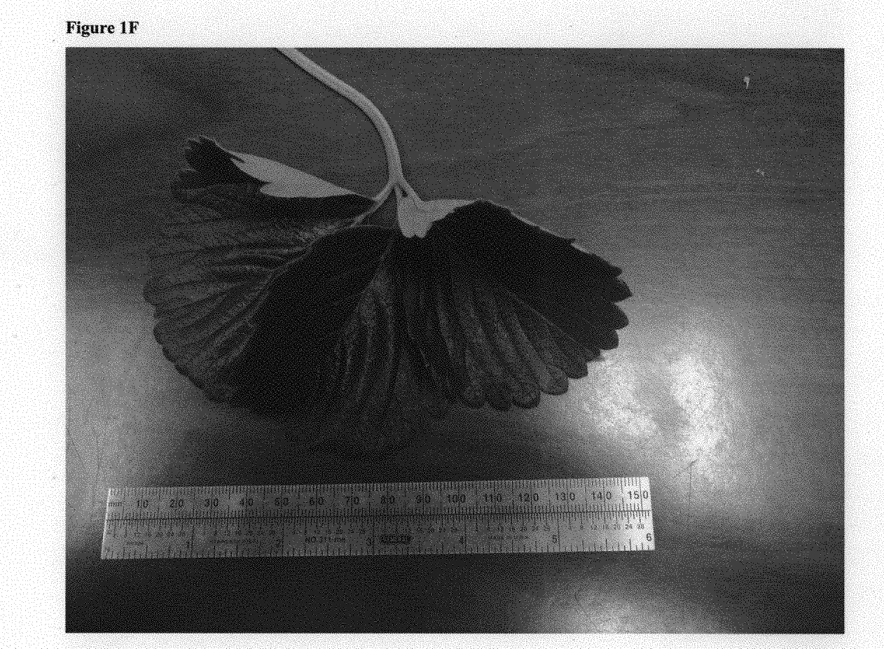

FIG. 1G shows lower leaf of ‘Caprice’.

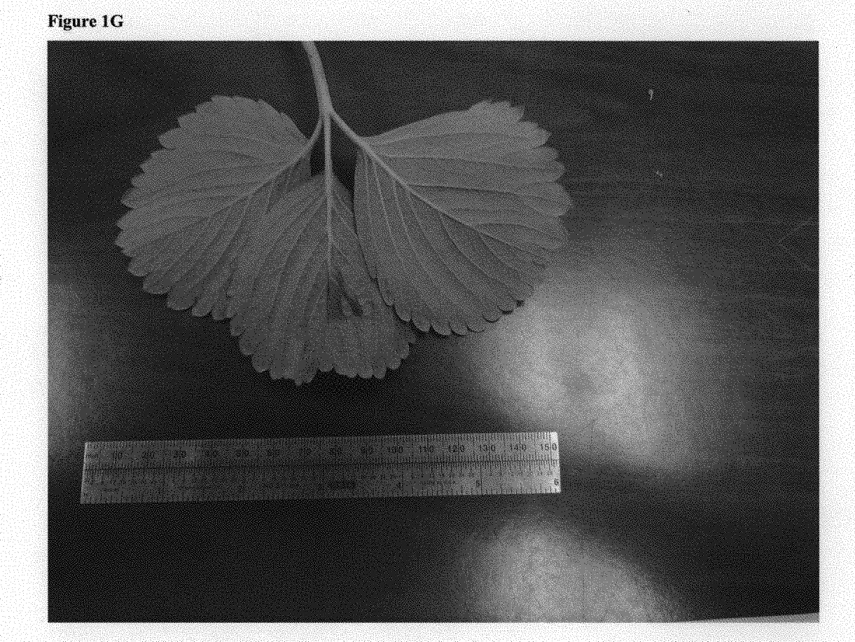

FIG. 2A shows ‘Victor’ plant about 4-month old.

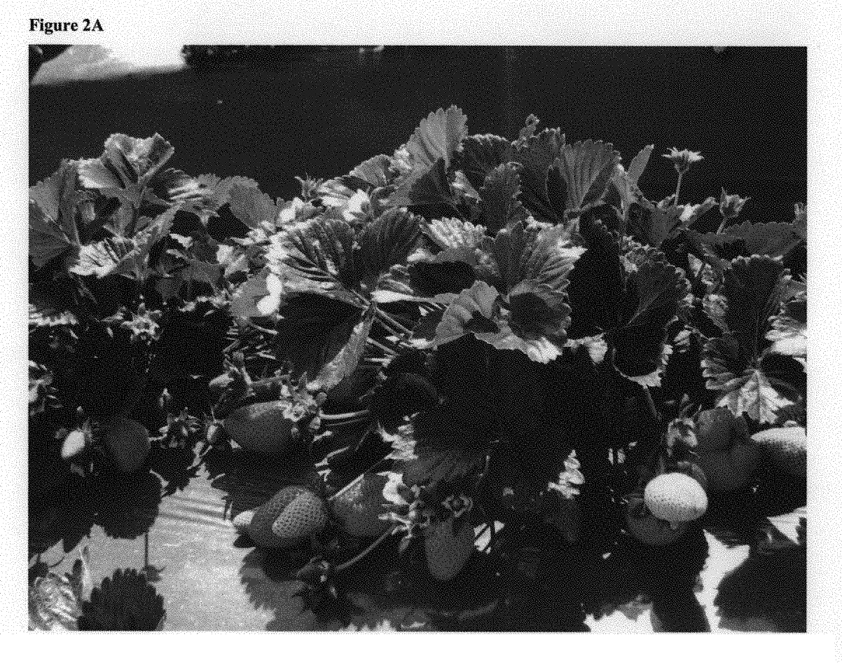

FIG. 2B shows ripe and near-ripe fruits of ‘Victor’ about 7-month old.

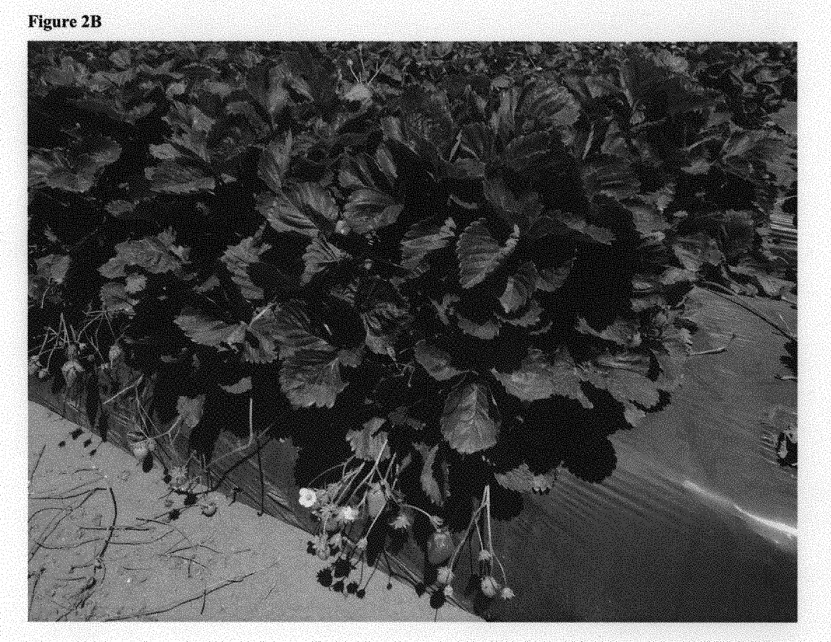

FIG. 2C shows flowers of ‘Victor’.

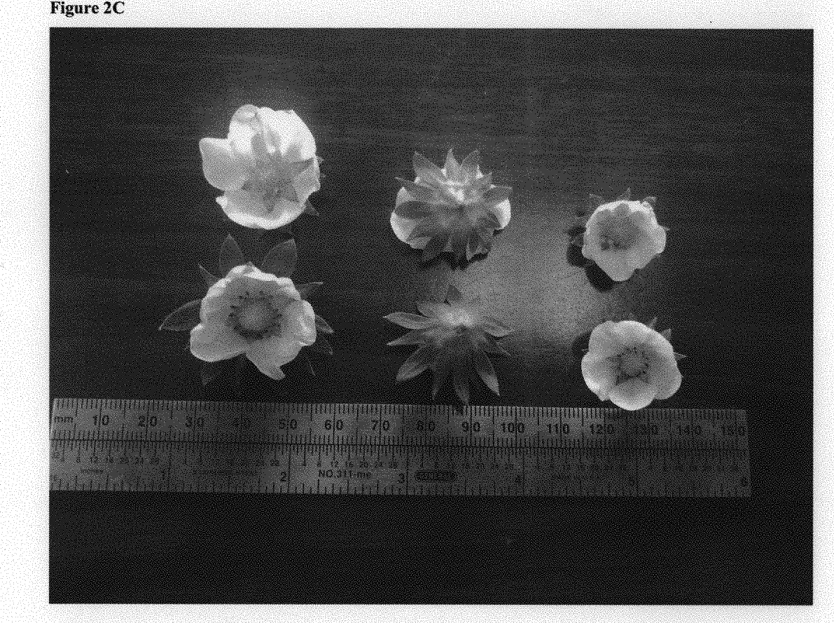

FIG. 2D shows fruits of ‘Victor’.

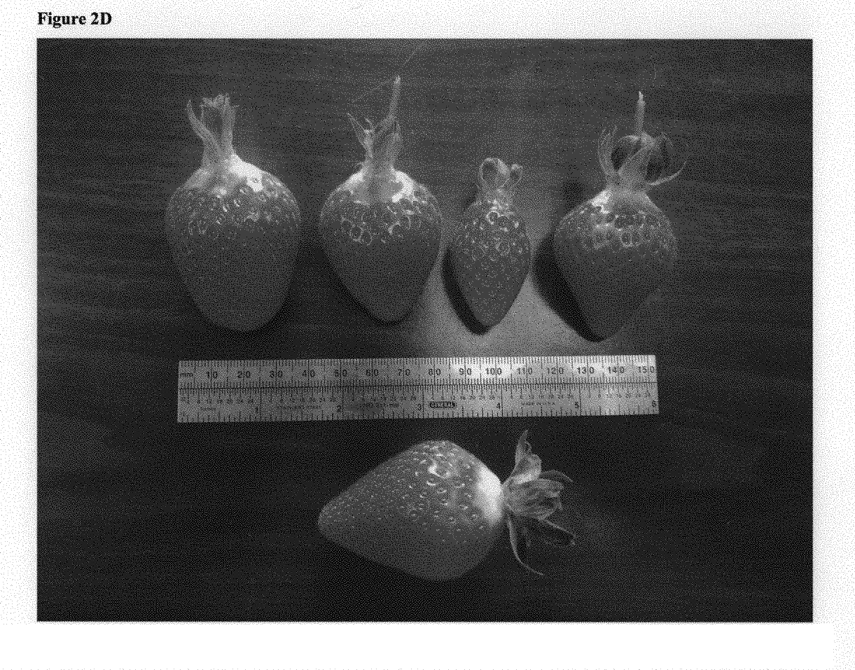

FIG. 2E shows cut fruits of ‘Victor’.

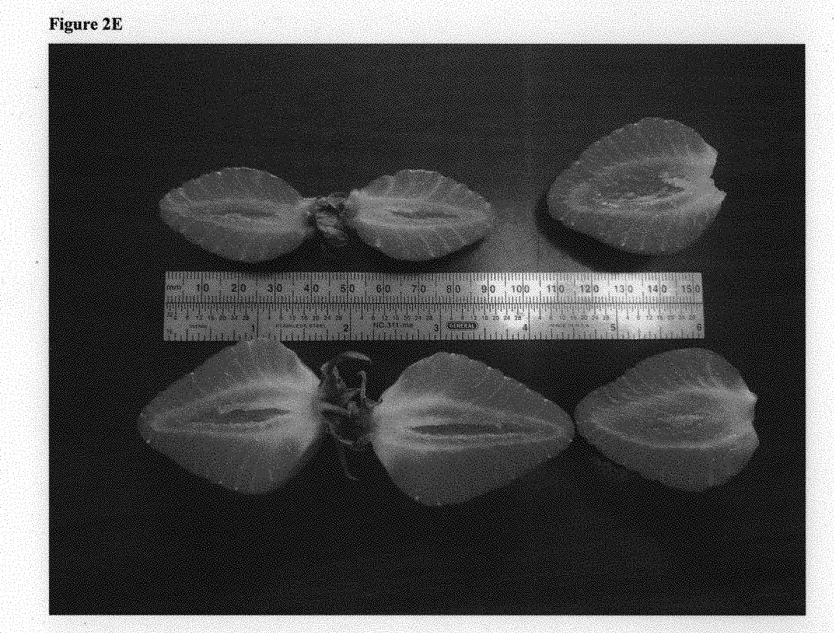

FIG. 2F shows upper leaf of ‘Victor’.

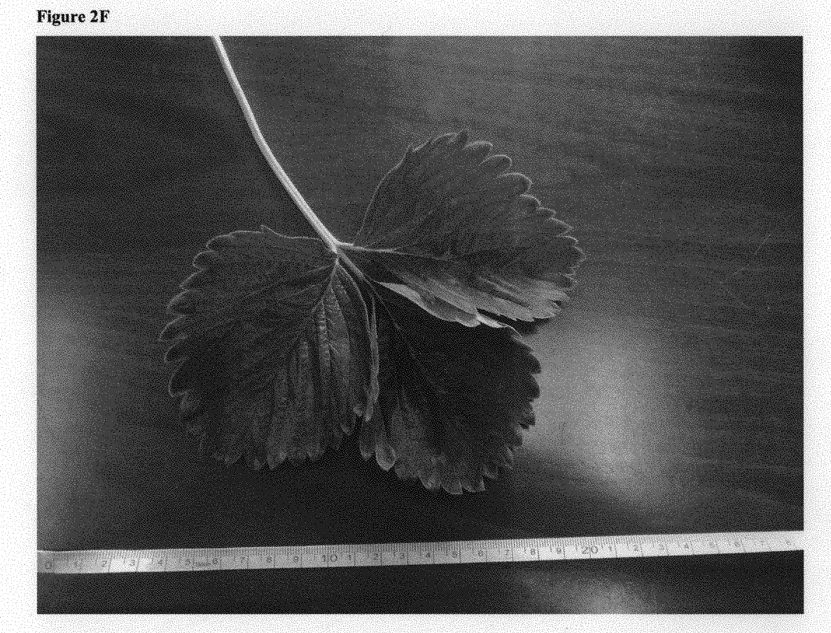

FIG. 2G shows lower leaf of ‘Victor’.

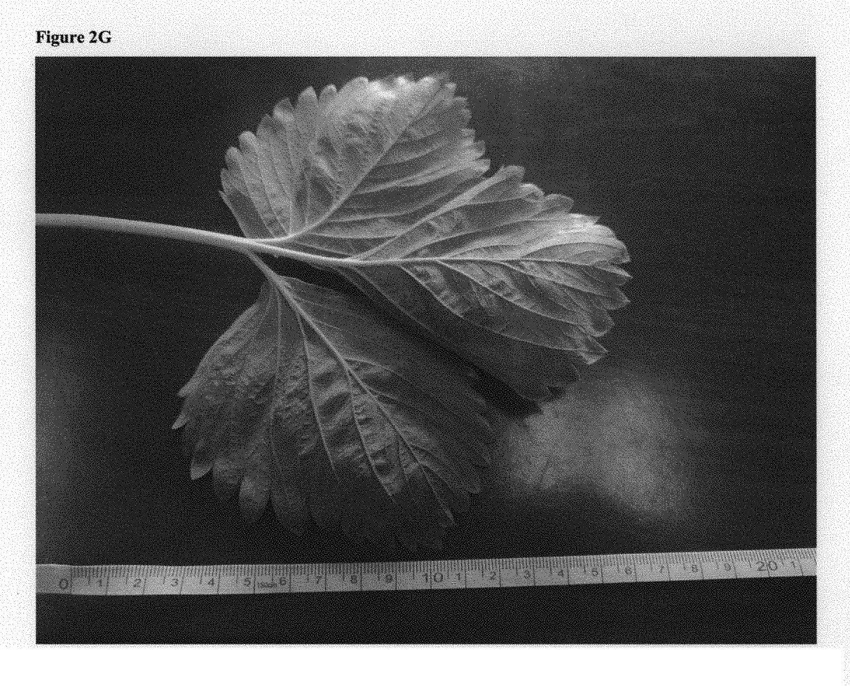

FIG. 2H shows petiole of ‘Victor’.

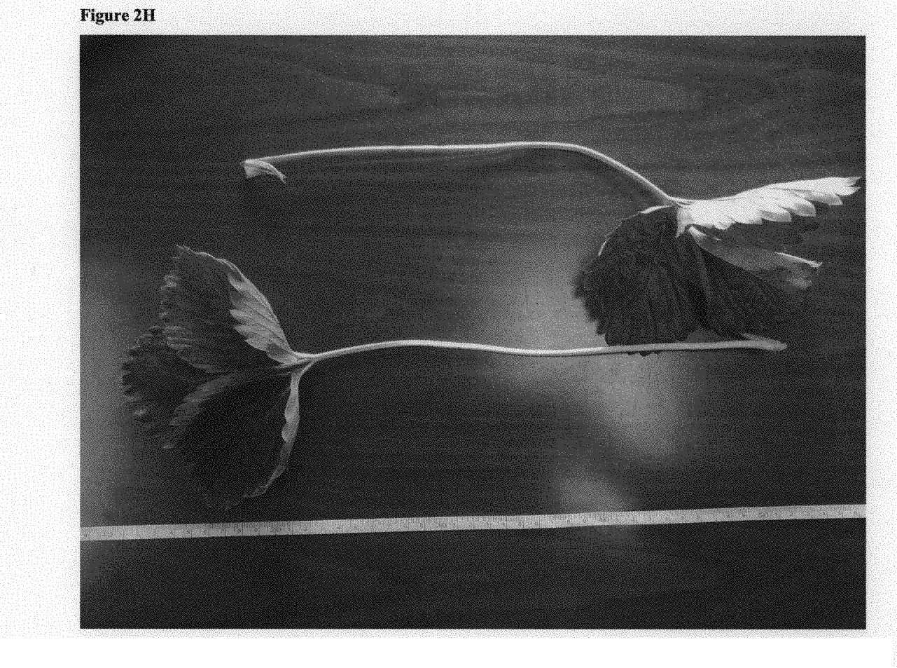

### DETAILED DESCRIPTION OF THE INVENTION

### ‘Caprice’ (a.k.a. ‘109862’)

The following traits in combination distinguish strawberry variety ‘Caprice’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

‘Caprice’ has not been observed under all possible environmental conditions, and the phenotype may vary significantly with variations in environment. The following observations, measurements, and comparisons describe this plant as grown under normal conditions in Watsonville, Calif. unless otherwise noted.

TABLE 1
‘Caprice’

| CharType | Characteristic | ‘Caprice’ |
|---|---|---|
| General | Plant Habit | annual |
| | Plant Growth Habit | upright |
| | Day length | Day Neutral |
| | Plant Height (cm) | 35 |
| | Plant Width (cm) | 32 |
| | Density of foliage | medium |
| | Plant vigor | moderate to high |
| Leaf | Terminal leaflet width (mm) | 70 to 80 |
| | Terminal leaflet length (mm) | 77.5 |
| | No. teeth/terminal leaflet: | 25 |
| | Shape of the terminal leaflet base | obtuse to rounded |
| | Shape of terminal leaflet in cross- | straight |
| | section | |
| | Margin description of the terminal | serrate to crenate |
| | Color of upper side of leaves | 137A |
| | Color of lower side of leaves | 137C |
| | Leaf blistering | weak |
| | Leaf glossiness | strong |
| Limbs | Petiole length (cm) | 26.5 |
| | Petiole diameter (mm) | 3.68 |
| | Petiole color | 145A |
| | Petiolule length (mm) | 1.5 |
| | Petiolule diameter (mm) | 2.32 |
| | Stipule length (cm) | 2.5 |
| | Stipule width (cm) | 0.7 |
| | Stipule anthocyanin | Present |
| | Stipule color (color code) | 149D |
| | Pedicel color (color code) | 145A |
| | attitude of hairs on | upwards |
| | petiole and pedicel | |
| Inflorescence | Inflorescence position relative to | above |
| | foliage | |
| | flower arragement of petals | touching |
| | Petal length (cm) | 1.3 |
| | Petal width (cm) | 1.5 |
| | Petl number per flower | 5 to 6 |
| | Upper Petal color | 155C |
| | Lower Petal color | N999D |
| | Peduncle size | medium |
| | Calyx diameter (cm) | 3 to 4 |
| | Corolla diameter (cm) | 2 |
| | Seapl length (cm) | 1.1 |
| | Sepal width (cm) | 0.7 |
| | Time of flowering | March |
| | (50% of plants in bloom) | |
| | Shape of stigma | capitate |
| | Color or stigma | 12A |
| | Length of style (mm) | 2 |
| | Color of style | 4A |
| | Color of the ovary | 145A |
| | Length of the stamens (mm) | 4 |
| | Number of stamen | 23 to 25 |
| | Shape of anther | dorsifixed |
| | Size of anther | medium |
| | Color of anther | 12A |
| | Amount of pollen | medium |
| | Color of pollen | 12A |
| | Color of filament | 149D |
| | Length of filament (mm) | 4 |
| | Number of flowers per truss | 3 to 6 |
| Stolon | Stolon number | 2 |
| | Stolon anthocyanin | 181A |
| | Widest diameter of stolon | 4.62 |
| | At leaf attachment (mm) | |
| | Stolon color | 145A |
| Fruit | Number of fruit per truss | 2 to 4 |
| | Fruit length (cm) | 34.1 |
| | Fruit width (cm) | 29.5 |
| | Fruit skin color | 41A |
| | Fruit fresh color excluding core | 44A |
| | Fruit core color | 41A |
| | Fruit weight (g) | 22.7 |
| | Predominant fruit shape | conic |
| | Shape difference between pri- | No shape difference |
| | mary & secondary fruits | |
| | Width of band without of achenes | narrow |
| | Fruit glossiness | medium |
| | Position of achenes | below surface |
| | Achene color | 145B |
| | Achenes per fruit | 288 |
| | Achene weight (g) | 0.17 |
| | Position of calyx | even |
| | level of adherence of calyz | medium |
| | Color of calyx | 136A |
| | Firmness of flesh | medium |
| | Evenness of flesh color | nearly even |
| | Sweetness (brix) | 10 |
| | pH | 3.45 |
| | Yield (g per plant per season) | 2791 |

### ‘Victor’ (a.k.a. ‘102850’)

The following traits in combination distinguish strawberry variety ‘Victor’ from the known strawberry varieties. Plants for the botanical measurements in the present application were grown as annuals. In the following description, color references are made to The Royal Horticultural Society Colour Chart, 1995 Edition, except where general terms of ordinary dictionary significance are used.

‘Victor’ has not been observed under all possible environmental conditions, and the phenotype may vary significantly with variations in environment. The following observations, measurements, and comparisons describe this plant as grown under normal conditions in Watsonville, Calif. unless otherwise noted.

TABLE 2
‘Victor’

| CharType | Characteristic | ‘Victor’ |
|---|---|---|
| General | Plant Habit | annual |
| | Plant Growth Habit | spreading to semi- |
| | | upright |
| | Day length | Short Day |
| | Planting season | Fall |
| | Height (cm) | 36 |
| | Width (cm) | 40 |
| | Density of foliage, vigor | light to medium |
| | Plant vigor | moderate |
| | Rain/weather tolerance | moderate |
| | Harvest Ease | easy to moderate |
| Leaf | Shape | concave |
| | Terminal leaflet width (cm) | 8.9 |
| | Terminal leaflet length (cm) | 9.1 |
| | Terminal leaflet length/width ratio | 1.02 |
| | Number of teeth/terminal leaflet | 22 to 24 |
| | Shape of the terminal leaflet base | obtuse |
| | Shape of terminal leaflet in cross- | concave |
| | section | |
| | Margin description of the terminal | serrate |
| | Shape of teeth/margin | serrate |
| | Color of upper side of leaves | 137A |
| | Color of lower side of leaves | 139C |
| | Leaf blistering | weak |
| | Leaf glossiness | medium |
| | Leaf variegation | absent |
| | Terminal Leaflet margin | flat |
| | Terminal Leaflet shape: | orbicular |
| | Terminal Leaflet shape of base | rounded |
| | Terminal Leaflet shape of apex | rounded |
| Limbs | Petiole length (cm) | 26 |
| | Petiole diameter (mm) | 3.43 |
| | Petiole pubescence | medium |
| | Petiole color | 145B |
| | Petiolule length (cm) | 1.8 |
| | Petiolule diameter (mm) | 2.3 |
| | Stipule length (cm) | 3.2 |
| | Stipule width (cm) | 1 |
| | Stipule pubescence | medium |
| | Stipule anthocyanin | absent |
| | Stipule color (color code) | 145C |
| | Pedicel color (color code) | 145A |
| | Attitude of hairs on petiole annd pedicel | horizontal |
| Inflorescence | Inflorescence position relative to | above |
| | foliage | |
| | Flower arragement of petals | touching |
| | Flower size | medium |
| | Petal shape | oval |
| | Petal apex | rounded |
| | Petal margin | entire |
| | Petal base shape | concave |
| | Petal length (cm) | 1.1 to 1.5 |
| | Petal width (cm) | 1.2 to 1.4 |
| | Petal length/width ratio | 0.91 to 1.07 |
| | Petal number per flower | 5 to 7 |
| | Upper Petal color (color code) | 155C |
| | Lower Petal color (color code) | 155C |
| | Peduncle size | medium |
| | Calyx diameter (cm) | 2.68 cm |
| | Corolla diameter (cm) | 1.8 to 2.9 |
| | Calyx diameter relative to corolla | larger to equal |
| | Inner calyx | same size |
| | Diameter relative to outer | smaller to equal |
| | Sepal shape | elliptical |
| | Sepal apex | convex |
| | Sepal margin | entire |
| | Sepal length (cm) | 1.3 |
| | Sepal width (cm) | 0.35 |
| | Sepal number per flower | 10 |
| | Receptacle color | 6A |
| | Fertility (self-fertile or self-infertile) | self-fertile |
| | Time of flowering (50% of plants in | March |
| | bloom) | |
| | Shape of stigma | capitate |
| | Color of stigma | 22A |
| | Length of style (mm) | 2 |
| | Color of style | 4A |
| | Color of the ovary | 145C |
| | Number of stamen | 26 |
| | Length of the stamens (mm) | 4 |
| | Shape of anther | dorsifixed |
| | Size of anther | medium |
| | Color of anther | 20A |
| | Amount of pollen | scarse |
| | Color of pollen | 12A |
| | Color of filament | 145C |
| | Length of filament mm | 3 |
| stolon | Stolon number | 7 to 32 |
| | Stolon anthocyanin | 184D |
| | Stolon thickness | small to medium |
| | Stolon pubescence | medium |
| | Widest diameter of stolon | 3.4 |
| | At leaf attachment (mm) | |
| | Stolon color | 145B |
| Fruit | Fruiting truss length (cm) | 12.1 |
| | Fruiting truss diameter (mm) | 5.19 |
| | Number of fruit per truss | 2 to 4 |
| | Fruiting truss attitude | outwards |
| | Fruit length (cm) | 5.21 |
| | Fruit width (cm) | 3.36 |
| | Fruit skin color | 44A to 45A |
| | Fruit flesh color excluding core | 44A |
| | Fruit core color | 41A |
| | Fruit length/width ratio | 1.55 |
| | Fruit weight (g) | 25.3 |
| | Relative fruit size | medium |
| | Predominant fruit shape | long-conic |
| | Shape difference between primary & | No shape differ- |
| | secondary fruits | ence |
| | Width of band without od achenes | broad |
| | Fruit glossiness | strong |
| | Position of achenes | below surface |
| | Achene color | 151D |
| | Achenes per fruit | 288 |
| | Achene weight (g) | 0.18 |
| | Position of calyx | above surface |
| | level of adherence of calyx | medium to strong |
| | Color of calyx | 137A |
| | Pose of calyx segments | reflexed |
| | Size of calyx in relation to fruit | same to smaller |
| | Firmness of flesh | medium to firm |
| | Evenness of flesh color | even |
| | Fruit hollow length (cm) | 2.7 |
| | Fruit hollow width (cm) | 0.6 |
| | Fruit hollow length/width ratio | 4.5 |
| | Hollow center | small |
| | Sweetness (Brix) | 9.5 |
| | Acidity (pH) | 3.6 |
| | Texture when tasted | medium |
| | Time of fruit ripening | April |
| | Harvest maturity (50% of plants with | April |
| | ripe fruit) | |
| | Type of bearing | Short Day |
| | Yield (grams per plant per season) | 2776 |
| | Firmness | firm |
| | Surface Texture | smooth |
| | Appearance Score (from 1-5, with 5 = | 4 |
| | best) | |
| | Cull rate: 1-Per cent Usable Fruit | 0.2 |

1. A new and distinct cultivar of strawberry plant named ‘Caprice’ substantially as shown and described herein.
